(19) United States
(12) Patent Application Publication
(10) Pub. No.: US 2021/0275575 A1
(43) Pub. Date: Sep. 9, 2021

(54) COMPOSITION FOR IMPROVING RENAL FUNCTION IN RENAL DISEASES CONTAINING MOLECULAR HYDROGEN

(71) Applicant: MiZ Company Limited, Kanagawa (JP)

(72) Inventors: Fumitake SATOH, Kanagawa (JP); Ryosuke KUROKAWA, Kanagawa (JP); Hideyuki SASAKI, Tokyo (JP); Yoshiaki SASAKI, Tokyo (JP)

(73) Assignee: MiZ Company Limited, Kanagawa (JP)

(21) Appl. No.: 17/188,617

(22) Filed: Mar. 1, 2021

(30) Foreign Application Priority Data
Mar. 3, 2020 (JP) 2020-035995

(51) Int. Cl. A61K 33/00; A61K 9/00; A61P 13/12

(52) U.S. Cl. CPC A61K 33/00; A61P 13/12; A61K 9/007

(57) ABSTRACT

The present application provides a composition for improving or preventing human renal diseases, containing molecular hydrogen as an active ingredient, for example, a composition for improving or preventing renal diseases in a human patient having acute or chronic nephritis, renal failure, and/or nephrotic syndrome, for example, for improving urinary protein and urinary protein/urinary creatinine ratio to a normal range, and a method for improving renal diseases by administering the composition to the patient.

### RELATED APPLICATIONS

This application claims priority to Japanese Patent Application No. 2020-035995, filed Mar. 3, 2020, which claims priority to Japanese Patent Application No. 2019-057964, filed Mar. 26, 2019, the entire contents of which are incorporated herein by reference.

### BACKGROUND

### 1. Field of the Invention

The present invention relates to a composition for improving renal function in renal diseases (for example, acute or chronic nephritis, renal failure, and/or nephrotic syndrome) in human patients by administering a composition containing molecular hydrogen. Specifically, the composition can improve renal disease in the patient, for example, improve urinary protein and urinary protein/urinary creatinine ratio to a normal range, or improve edema (also referred to as “swelling”).

### 2. Description of the Related Art

The kidneys have the role of filtering waste products in the blood and eliminating them from the body as urine, as well as the role of regulating the concentration and amount of urine to keep the body's water content constant, secreting erythropoietin to promote the production of red blood cells in the bone marrow, secreting renin from the kidneys to raise the blood pressure when the blood pressure decreases and the renal blood flow decreases, and activating vitamin D to convert it to active vitamin D.

The above renal function is reduced due to, for example, high blood pressure, diabetes, infectious diseases (cystitis, etc.), fatigue accumulation, smoking, etc., and becomes acute kidney disease, then chronic kidney disease (CKD) as it becomes chronic.

In chronic kidney disease, there is a decrease in renal function represented by the glomerular filtration rate (GFR), or the findings suggestive of kidney damage, typically urinary abnormalities such as proteinuria, image abnormalities such as only one kidney and polycystic kidney disease, blood abnormalities, and pathological findings, are chronically persistent (Evidence-based Clinical Practice Guideline for CKD 2018, Japanese Society of Nephrology; Non-Patent Literature 1).

In addition, diabetic nephropathy is known to develop about 10 years or more after contracting diabetes. Albuminuria (proteinuria) is observed in this disease, which produces a decrease in renal function and may lead to nephrotic syndrome.

When chronic nephritis further progresses and the mesh of the “glomerulus” which filters blood becomes clogged, renal function further decreases and waste products can no longer be sufficiently eliminated, which results in renal failure.

Chronic glomerulonephritis is one of the chronic kidney diseases, and for example, steroid preparations, immunosuppressants, dipyridamole (a drug reducing urinary protein) and the like are used as therapeutic agents. In addition, when there are symptoms of hypertension or hyperglycemia, for example, hypotensive agents, drugs for lowering blood sugar level, or the like are used.

Furthermore, it has been reported that kidney damage is suppressed when hydrogen dissolved water (HW) is administered to a rat model of chronic kidney disease (in this model, oxidative stress is given by administration of oxycarbonyl) (B. Chen et al., J Bioprocess Biotech 2018, 8:4 DOI: 10.4172/2155-9821.1000331; Non-Patent Literature 2). In this literature, urinary protein was also measured, but it was concluded that there was no difference from the control.

It has been pointed out that hydrogen can reduce reactive oxygen species (ROS), which are a major cause of oxidative stress in the body, and therefore may help alleviate the symptoms of diseases caused by oxidative stress (Ohta, S. (2015) Journal of Japanese Biochemical Society 87(1): 82-90; Non-Patent Literature 3).

The object of the present invention is to provide a composition for improving renal function in renal diseases.

### SUMMARY

The present inventors found that by administering a composition containing (or comprising) molecular hydrogen as an active ingredient to a human patient with renal disease (sometimes simply referred to as “human patient”), for example, it is possible to improve urinary protein and urinary protein/urinary creatinine ratio to a normal range, or improve edema (or swelling) in the patient, thereby completing the present invention.

Therefore, the present invention includes the following features.

(1) A composition for improving renal disease in humans, comprising molecular hydrogen as an active ingredient.

(2) The composition according to (1), wherein the improvement of the renal disease is an improvement of acute or chronic nephritis, an improvement of renal failure, and/or an improvement of nephrotic syndrome.

(3) The composition according to (2), wherein the acute or chronic nephritis is selected from the group consisting of glomerulonephritis, interstitial nephritis, pyelonephritis, acute progressive glomerulonephritis, anti-glomerular basement membrane antibody nephritis, membranoproliferative glomerulonephritis, endocapillary proliferative glomerulonephritis, crescentic glomerulonephritis, and diabetic nephritis.

(4) The composition according to (2) or (3), wherein the improvement of the acute or chronic nephritis comprises improving urinary protein and urinary protein/urinary creatinine ratio to a normal range, or improving edema.

(5) The composition according to (4), wherein the normal range of urinary protein is less than 0.2 g/day, and the normal range of the urinary protein/urinary creatinine ratio is less than 0.3 g/gCr.

(6) The composition according to any one of (1) to (5), wherein the composition is a gas comprising molecular hydrogen at a concentration greater than zero (0) and equal to 18.5 vol % or less.

(7) The composition according to any one of (1) to (5), wherein the composition is a biocompatible solution comprising an effective amount of molecular hydrogen.

(8) The composition according to (7), wherein the biocompatible solution is water, a beverage, saline, or an infusion.

(9) The composition according to (7) or (8), wherein the concentration of molecular hydrogen dissolved in the biocompatible solution is 1 to 10 ppm.

(10) A method for improving a renal disease, comprising administering the composition according to any one of (1) to (9) to a human patient having a renal disease and improving the renal disease in the patient.

(11) The method according to (10), wherein the improvement of the renal disease is an improvement of acute or chronic nephritis, an improvement of renal failure, and/or an improvement of nephrotic syndrome.

(12) The method according to (11), wherein the acute or chronic nephritis is selected from the group consisting of glomerulonephritis, interstitial nephritis, pyelonephritis, acute progressive glomerulonephritis, anti-glomerular basement membrane antibody nephritis, membranoproliferative glomerulonephritis, endocapillary proliferative glomerulonephritis, crescentic glomerulonephritis, and diabetic nephritis.

(13) The method according to (11) or (12), wherein the improvement of the acute or chronic nephritis comprises improving urinary protein and urinary protein/urinary creatinine ratio to a normal range, or improving edema.

### Advantageous Effects of Invention

According to the present invention, the composition containing molecular hydrogen as an active ingredient allows to improve renal diseases including, as an example, a renal disease derived from human diabetes and/or hypertension, for example, to improve urinary protein and urinary protein/urinary creatinine ratio to a normal range, to improve edema, and the like, by administering the composition to a human patient having renal disease (for example, intractable kidney diseases such as acute or chronic nephritis, renal failure, and/or nephrotic syndrome).

### DETAILED DESCRIPTION OF THE PREFERRED EMBODIMENTS

The present invention is further described in detail.

### 1. Renal Disease

Examples of the renal diseases in the present invention include nephritis (primary glomerular disease, secondary glomerular disease), nephrotic syndrome, acute kidney injury, chronic kidney disease, (chronic) renal failure, renal tumor, hereditary renal diseases, kidney/urinary tract stone disease, retroperitoneal fibrosis, renal amyloidosis, renal infarction, dysregulation of serum sodium (hypernatremia, hyponatremia), syndrome of inappropriate antidiuretic hormone secretion, dysregulation of serum potassium (hyperkalemia, hypokalemia), dysregulation of serum calcium (hypercalcemia, hypocalcemia), dysregulation of serum phosphate (hyperphosphatemia, hypophosphatemia), dysregulation of acid-base balance, uremia, and diseases related thereto.

Examples of primary glomerulonephritis include minimal change disease, focal glomerulosclerosis, mesangial proliferative glomerulonephritis (IgA nephropathy, non-IgA nephropathy), membranous nephropathy, endocapillary proliferative glomerulonephritis, endocapillary proliferative glomerulonephritis, crescentic glomerulonephritis, membranoproliferative glomerulonephritis, sclerosing glomerulonephritis, and diseases related thereto.

Examples of secondary glomerular disease include lupus nephritis, polyarteritis nodosa, microscopic polyangiitis, diabetic nephropathy, nephrosclerosis, and diseases related thereto.

Furthermore, examples of renal disease in the present invention include nephrotic syndrome (a disease in which protein in the blood is reduced due to the excretion of protein in urine, leading to hypoproteinemia, and resulting in swelling (edema)) and side effects of drugs used in the improvement of various renal diseases.

### 2. Nephritis

A typical primary renal disease is nephritis, and depending on the site of inflammation, for example, glomerulonephritis, interstitial nephritis, pyelonephritis, acute progressive glomerulonephritis, anti-glomerular basement membrane antibody nephritis, membranoproliferative glomerulonephritis, endocapillary proliferative glomerulonephritis, crescentic glomerulonephritis, diabetic nephritis, and the like are known. “Nephritis” in the present description includes the above-exemplified diseases. In addition, nephritis is divided into acute and chronic diseases according to the degree of symptom progression: acute when the symptoms progress rapidly, and chronic when the symptoms progress gradually.

Chronic nephritis refers to nephritis in which proteinuria, then as the disease progresses, hematuria persist over a long time (at least one year or more). For example, in the case of chronic glomerulonephritis, the kidney is damaged due to chronic inflammation of the filter called the glomerulus of the kidney, and as a result, proteins and red blood cells in the blood become mixed with urine. Renal function then gradually declines as the symptoms become chronic, eventually leading to renal failure. The initial symptoms of chronic nephritis are the occurrence of swelling (edema) in the feet, hands, face, and the like. This is because when the glomerulus of the kidney, which filters blood, is damaged, the mesh of the glomerulus becomes clogged, which impairs blood filtration and deteriorates the function of eliminating waste products, excess water, and salt from the body.

In chronic nephritis, either or both of the following (a) and (b) persists for 3 months or longer (Evidence-based Clinical Practice Guideline for CKD 2018, Japanese Society of Nephrology; Non-Patent Literature 1).

(a) The presence of kidney damage is apparent through urine, diagnostic imaging, blood, and pathological examination, and in particular, urinary abnormalities, that is, a proteinuria of 0.15 g/gCr or more (albuminuria of 30 mg/gCr or more) are observed.

(b) GFR<60 ml/min/1.73 m². Here, GFR is calculated using the following GFR estimation formula for Japanese from serum creatinine (Cr) level, gender, and age (here, eGFR is estimated GFR): eGFR (ml/min/1.73 m²)=194×serum Cr (mg/dl)−1.094 x age (years)−0.287 (however, in the case of women, multiply by 0.739).

The Cr level can be measured, for example, by an enzymatic method. Specifically, in this method, creatinine is converted to creatine by creatinase, which then produces sarcosine by creatinase, and then hydrogen peroxide is produced by sarcosine oxidase. Next, the quinone pigments produced from various chromogens in the coexistence of peroxidase are quantified. Here, sodium azide, which is an inhibitor of catalase, is used to prevent the decomposition of hydrogen peroxide derived from creatinine.

Renal function can usually be measured by blood urea nitrogen (BUN), serum creatinine, diagnostic imaging, renal biopsy, and the like (for example, Japan Association of Kidney Disease Patients; www.zjk.or.jp/kidney-disease/inspection-method/index.html).

Urea nitrogen is a waste product produced after protein is utilized, and is filtered by the glomerulus of the kidney, then eliminated in the urine. However, when renal function decreases, it is not completely filtered and accumulates in the blood, which increases the level of urea nitrogen in the blood. Normal blood urea nitrogen levels are 20 mg/dl or less.j

Creatinine is a waste product of protein contained in muscle. Originally, like urea nitrogen, it is filtered by the glomerulus of the kidney and eliminated in the urine, but when the function of the kidney decreases, the amount eliminated in the urine decreases and creatinine accumulates in the blood. Serum creatinine levels increase with decreased renal function. Normal serum creatinine levels are 1.2 mg/dl or less for men and 1.0 mg/dl or less for women.

Diagnostic imaging is, for example, an ultrasound or an abdominal CT scan, and examines the shape and size of the kidney, and the presence of complications (tumor, stones, etc.).

In renal biopsy, a portion of kidney tissue is removed and examined under a microscope to make a clear diagnosis and determine the most appropriate treatment for abnormalities in renal function, such as chronic proteinuria or hematuria.

Regarding urinary protein, patients with urinary protein of 1+ or higher are at higher risk of not only developing end-stage kidney disease (ESKD), but also of cardiovascular death and all deaths compared to patients with −or ±.

In addition, the urinary protein/urinary Cr ratio (g/gCr) is calculated by quantifying urinary protein and measuring urinary Cr. A1 (normal) is less than 0.15 g/gCr, A2 (mild proteinuria) is 0.15 to 0.49 g/gCr and A3 (severe proteinuria) is 0.5 g/gCr or more. In the qualitative evaluation of urinary protein by urinary test strip, − is A1, ± is A2, and 1+ or more is A3. Furthermore, a urinary protein of 1+ is analyzed as corresponding to microalbuminuria (ACR 30 to 299 mg/g), and a urinary protein of 2+ or more as overt albuminuria (ACR 300 mg/g or more).

Furthermore, in the present invention, chronic nephritis includes secondary diabetic nephropathy.

Diabetes is a disease in which blood sugar levels rise mainly due to a lack of insulin secreted by the pancreas, resulting in various complications. When developing diabetes and continuously having poor glycemic control, complications usually develop in the order of the three major microvascular complications: neurosis, retinopathy, and nephropathy. Although it takes 10 to 20 years for nephropathy to appear, microalbuminuria (nephropathy stage 2) then proteinuria (nephropathy stage 3) appear early, often resulting in nephrotic syndrome. Renal function then gradually decreases, leading to renal failure.

In patients with diabetic nephropathy, blood sugar, hemoglobin A1c (HbA1c), and glyco-(glycated) albumin are measured by a diabetes test, and are all higher than normal levels. Moreover, serum creatinine, serum cystatin C, eGFR, uric acid, and the like are measured by a renal function test. eGFR is lower, but the others are higher.

In the present description, chronic nephritis is a disease in which either or both of the above (a) and (b) persists for 3 months or longer, and includes diabetic nephropathy (having diabetic symptoms and chronic nephritis symptoms).

### 3. Composition

The composition of the present invention contains molecular hydrogen as an active ingredient, and is used to improve renal disease in a human patient having renal disease, for example, to improve urinary protein and urinary protein/urinary creatinine ratio to a normal range.

In the present description, “renal disease” includes diseases as described in the above Sections 1 and 2, and is preferably an intractable kidney disease such as acute or chronic nephritis, renal failure, and/or nephrotic syndrome.

In the present description, “intractable kidney disease” refers to a kidney disease for which the improvement or recovery is impossible or difficult even with all the medicaments manufactured and sold by the modern medical and pharmaceutical industries, and for which the treatment methods have little evidence and are not established.

The composition of the present invention can improve the decrease in renal function in intractable renal diseases by administering it to a subject (preferably a human).

The normal range of urinary protein is less than 0.2 g/day, and the normal range of the urinary protein/urinary creatinine ratio is less than 0.3 g/gCr.

The composition of the present invention may be in the form of a medicament, or in the form of a food or drink (for example, a beverage). In any form, it should contain the effective concentration or effective amount of molecular hydrogen shown below.

In the present description, “hydrogen”, which is the active ingredient of the composition of the present invention, is molecular hydrogen, and is simply referred to as “hydrogen” or “molecular hydrogen” unless otherwise specified. Moreover, the term “hydrogen” used in the present description refers to, in molecular formula, H₂, D₂ (deuterium), HD (hydrogen deuteride), or a gas mixture thereof. D₂ is costly, but is known to have a stronger superoxide scavenging effect than H₂. The hydrogen usable in the present invention is H₂, D₂ (deuterium), HD (hydrogen deuteride), or a gas mixture thereof, preferably H₂, or, instead of H₂ or mixed with H₂, D₂ and/or HD may be used.

A preferred form of the composition of the present invention is a gas containing an effective amount of molecular hydrogen and air or oxygen, or a biocompatible liquid (also referred to as “biocompatible solution”) containing an effective amount of dissolved molecular hydrogen. These are referred to as “hydrogen-containing gas” and “hydrogen-dissolved liquid,” respectively.

The hydrogen-containing gas is preferably air containing molecular hydrogen or a gas mixture containing molecular hydrogen and oxygen gas (sometimes simply referred to as “oxygen”). The concentration of hydrogen in the hydrogen-containing gas is greater than zero (0) and equal to about 18.5 vol % or less, for example, about 0.1 to 18.5 vol %, for example, 1 to 10 vol %, for example, 2 to 10 vol %, 2 to 9 vol %, 2 to 8 vol %, 3 to 10 vol %, 3 to 9 vol %, 3 to 8 vol %, 3 to 7 vol %, 3 to 6 vol %, 4 to 10 vol %, 4 to 9 vol %, 4 to 8 vol %, 4 to 7 vol %, 4 to 6 vol %, 4 to 5 vol %, 5 to 10 vol %, 5 to 9 vol %, 5 to 8 vol %, 5 to 7 vol %, 6 to 10 vol %, 6 to 9 vol %, 6 to 8 vol %, 6 to 7 vol %, and the like. In the present invention, the higher the hydrogen concentration below the explosion limit, the longer the hydrogen administration period, or the longer the hydrogen administration time/dose, the greater the effect of improving renal disease in patients with renal disease tends to be.

In this regard, in conventional hydrogen gas inhalation therapy, the improvement effect on the disease (carcinoma) is finally exhibited by a hydrogen gas at a high concentration of 66% or 99%. However, in the present invention, it is preferable to incorporate hydrogen to the composition of the present invention and administer it to a subject such as a human under conditions safe for the subject, and even if the hydrogen has a low concentration greater than 0 (zero) and equal to 18.5 vol % or less, a sufficient improving effect on renal disease can be exhibited. In addition, since hydrogen is a flammable and explosive gas, it is preferable to administer it to a patient by incorporating hydrogen to the composition of the present invention under conditions safe for human patients when improving renal diseases.

When the gas other than molecular hydrogen is air, the concentration of air is, for example, in the range of 81.5 to 99.9 vol %.

When the gas other than molecular hydrogen is a gas containing oxygen gas, the concentration of oxygen gas is, for example, in the range of 21 to 99.9 vol %.

As other gases, nitrogen gas can be contained, for example.

Specifically, the hydrogen-dissolved liquid (also referred to as “hydrogen-containing liquid”) is a biocompatible solution in which molecular hydrogen is dissolved. Here, examples of the biocompatible solution include, but are not limited to, water (for example, purified water, sterilized water), saline, infusions (for example, drip solutions, Ringer's solutions, and the like, which may contain a therapeutic drug), injection solutions, beverages (for example, tea drinks such as green tea and black tea, green juices, vegetable juices, fruit juices, and the like).

The hydrogen concentration in the hydrogen-dissolved liquid may be, but is not limited to, for example, 1 to 10 ppm, or more, preferably 1.2 to 8 ppm, such as 1.5 to 7 ppm, 1.5 to 5 ppm, 2 to 8 ppm, 2 to 7 ppm, 2 to 6 ppm, 2 to 5 ppm, 3 to 8 ppm, 3 to 7 ppm, 4 to 8 ppm, and 5 to 8 ppm, and more preferably 3 to 8 ppm, such as 3 to 7 ppm, 4 to 8 ppm, and 5 to 8 ppm. In the present invention, the higher the concentration of dissolved hydrogen below the explosion limit, the greater the effect of improving renal disease in patients with renal disease, for example, improving the urinary protein and urinary protein/urinary creatinine ratio to a normal range.

The hydrogen-containing gas or the hydrogen-dissolved liquid is blended to achieve a predetermined hydrogen concentration, then filled, for example, in a pressure-resistant container (for example, a stainless steel cylinder, an aluminum can, preferably a pressure-resistant plastic bottle (for example, a pressure-resistant PET bottle) and a plastic bag, an aluminum bag, or the like laminated with an aluminum film on the inside). Aluminum has the property of allowing few hydrogen molecules to permeate. Alternatively, at the time of administration, the hydrogen-containing gas or the hydrogen-dissolved liquid may be produced on the spot using an apparatus such as a hydrogen generator, a hydrogen water generator, or a hydrogen addition apparatus, for example, a known or commercially available hydrogen supplying apparatus (apparatus for generating hydrogen-containing gas), a hydrogenation device (apparatus for generating hydrogen water), a nondestructive hydrogen adding apparatus (for example, an apparatus for non-destructively adding molecular hydrogen to the inside of a biocompatible solution bag such as a drop solution).

Alternatively, for a hydrogen-containing gas or a hydrogen-dissolved liquid (or a hydrogen-containing liquid), purified hydrogen gas may be directly injected into a biocompatible liquid such as water, a beverage, saline, or an infusion to produce a hydrogen-dissolved liquid of a predetermined concentration, or purified hydrogen gas and purified air or oxygen gas may be directly mixed to produce a hydrogen-containing gas of a predetermined concentration.

The hydrogen supplying apparatus allows to mix molecular hydrogen generated by the reaction of a hydrogen generating agent (for example, metal aluminum, magnesium hydride, and the like) and water, with a diluting gas (for example, air, oxygen, and the like) at a predetermined ratio (Japanese Patent No. 5228142, and the like). Alternatively, molecular hydrogen generated by the electrolysis of water is mixed with a diluting gas such as oxygen or air (Japanese Patent No. 5502973, Japanese Patent No. 5900688, and the like). This allows to prepare a hydrogen-containing gas with a hydrogen concentration in the range of 0.1 to 18.5 vol %.

A hydrogenation device is an apparatus that generates hydrogen using a hydrogen generating agent and a pH adjuster, and dissolves it in a biocompatible solution such as water (Japanese Patent No. 4756102, Japanese Patent No. 4652479, Japanese Patent No. 4950352, Japanese Patent No. 6159462, Japanese Patent No. 6170605, Japanese Patent Laid-Open No. 2017-104842, Japanese Patent No. 6159462, and the like). Examples of combinations of the hydrogen generating agent and the pH adjuster include metal magnesium and a strongly acidic ion exchange resin or an organic acid (for example, malic acid, citric acid, and the like), and metal aluminum powder and calcium hydroxide powder. This allows to prepare a hydrogen-dissolved liquid with a dissolved hydrogen concentration of about 1 to 10 ppm (for example, trade name “7Water” (Quasia), and the like).

A nondestructive hydrogen adding apparatus is an apparatus or device for adding hydrogen molecules to a commercially available biocompatible solution such as a drip solution (for example, sealed in a hydrogen-permeable plastic bag such as a polyethylene bag) from the outside of the package, and is commercially available from, for example, MiZ Company Limited (www.e-miz.co.jp/technology.html). This apparatus can immerse a bag containing a biocompatible solution in saturated hydrogen water to permeate hydrogen in the bag and aseptically dissolve hydrogen in the biocompatible solution until reaching a concentration equilibrium. The apparatus is composed of, for example, an electrolysis tank and a water tank, and the water in the water tank circulates through the electrolysis tank and water tank, which allows to generate hydrogen by electrolysis. Alternatively, a simplified disposable device can be used for the same purpose (Japanese Patent Laid-Open No. 2016-112562, and the like). This device contains a biocompatible solution containing-plastic bag (a hydrogen-permeable bag, for example, a polyethylene bag) and a hydrogen generating agent (for example, metal calcium, metal magnesium/cation exchange resin, and the like) in an aluminum bag, and the hydrogen generating agent is wrapped in, for example, a nonwoven fabric (for example, a water vapor permeable nonwoven fabric). The hydrogen generated by wetting the hydrogen generating agent wrapped in the nonwoven fabric with a small amount of water such as water vapor permeates the plastic bag and is dissolved nondestructively and aseptically in the biocompatible solution.

The hydrogen-containing gas or hydrogen-dissolved biocompatible solution (for example, water (for example, purified water, sterilized water), a beverage, saline, an infusion (including a drip solution and Ringer's solution), an injection solution, a beverage, and the like), prepared using the above apparatuses or devices, can be administered orally or parenterally to a human patient having renal disease.

Other forms of the composition of the present invention include dosage forms (for example, tablets, capsules, and the like) containing a hydrogen generating agent capable of generating hydrogen in the gastrointestinal tract and that is prepared for oral administration (or ingestion) to human patients. It is preferable that the hydrogen generating agent be constituted by components approved as a food or food additive, for example.

As a method for administering the composition of the present invention to a human patient, pulmonary administration by inhalation, aspiration or the like is preferable when using molecular hydrogen as an active ingredient, and oral administration or intravenous administration (including drip) is preferable when using a hydrogen-dissolved liquid as an active ingredient. When inhaling a gas, the gas is inhaled through the mouth or nose via a nasal cannula or a mask-type device covering the mouth and nose, then sent to the lungs to be delivered to the whole body through the blood.

As for the hydrogen-dissolved liquid to be orally administered, for example, a liquid stored at a low temperature and cooled or a liquid stored at ordinary temperature may be administered to a human patient. Hydrogen is known to dissolve in water at a concentration of about 1.6 ppm (1.6 mg/L) at ordinary temperatures and pressures, and the difference in solubility with temperature to be relatively small. Alternatively, when in the form of a drip solution or an injection containing molecular hydrogen prepared using the above nondestructive hydrogen adding apparatus, for example, the hydrogen-dissolved liquid may be administered to a human patient through a parenteral route of administration such as intravenous administration and intraarterial administration.

The hydrogen-containing gas having the above hydrogen concentration or the hydrogen-dissolved liquid having the above dissolved hydrogen concentration can be administered to a human patient having renal disease (for example, acute or chronic nephritis, renal failure, and/or nephrotic syndrome), for example, once or a plurality of times (for example, 2 to 3 times) per day, for example, for a time period of 1 to 6 months or more, or 1 to 3 years or more. The hydrogen-containing gas can be administered over a period of, for example, 1 to 2 hours or more, for example, 2 to 5 hours per administration. Moreover, the hydrogen-containing gas can be administered to a human patient by pulmonary administration through inhalation or aspiration under an atmospheric pressure environment or, for example, under a high atmospheric pressure environment in a range exceeding standard atmospheric pressure (about 1.013 atm) and 7.0 atm or less, for example, 1.02 to 7.0 atm, preferably 1.02 to 5.0 atm, more preferably 1.02 to 4.0 atm, and further preferably 1.02 to 1.35 atm. The high atmospheric pressure environment can contain, for example, the hydrogen-containing gas (hydrogen-containing oxygen or hydrogen-containing air) inside.

When treating to improve renal disease in a patient having renal disease (for example, acute or chronic nephritis, renal failure, and/or nephrotic syndrome), for example, to improve urinary protein and urinary protein/urinary creatinine ratio to a normal range by the composition of the present invention, it is desirable to use a hydrogen generator, a hydrogen water generator, or a hydrogen adding apparatus (for example, an apparatus such as the above hydrogen supplying apparatus (or hydrogen breathing apparatus), hydrogenation device (or hydrogen water generator), or nondestructive hydrogen adding apparatus (apparatus for nondestructively dissolving molecular hydrogen in a biocompatible solution such as a drip solution sealed in a hydrogen permeable bag), for which a sufficient therapeutic effect and safety has been confirmed.

The present invention further provides a method for improving renal diseases, including administering the composition of the present invention containing molecular hydrogen as an active ingredient to a human patient having renal disease (for example, acute or chronic nephritis, renal failure, and/or nephrotic syndrome) to improve renal disease (or improve the decrease in function due to the renal disease), for example, to improve urinary protein and urinary protein/urinary creatinine ratio to a normal range, to improve edema, and the like in the patient.

Examples of the decrease in function due to renal disease include a decrease in the ability to filter blood and eliminate waste products as urine, a decrease in glomerular function, and a decrease in hormones produced by the kidney (for example, erythropoietin, renin, and the like).

The acute or chronic nephritis is not limited and, for example, can be selected from the group consisting of glomerulonephritis, interstitial nephritis, pyelonephritis, acute progressive glomerulonephritis, anti-glomerular basement membrane antibody nephritis, membranoproliferative glomerulonephritis, endocapillary proliferative glomerulonephritis, crescentic glomerulonephritis, and diabetic nephritis.

The effective amount of molecular hydrogen is difficult to determine easily, but is the amount of gas containing a high concentration of molecular hydrogen (for example, a molecular hydrogen concentration of 4 to 10 vol %) or of biocompatible solution containing a high concentration of molecular hydrogen (for example, a molecular hydrogen concentration of 4 to 10 ppm) prepared at the time of use, inhaled or ingested for about 1 to 5 hours/day for a gas and about 1 to 3 L/day for water, and is inhaled or ingested for a period of, for example, about 3 months to about 2 years or more while following the progress of the improvement of the symptoms of renal disease.

The route of administration of the composition of the present invention is oral administration or parenteral administration, and examples of parenteral administration include nasal inhalation and intravenous administration. In the case of intravenous administration, for example, a nondestructive hydrogen adding apparatus is used to aseptically dissolve molecular hydrogen in an infusion, and then the hydrogen-dissolved solution is administered.

The composition, renal diseases, nephritis, renal failure, normal range of urinary protein and urinary protein/urinary creatinine ratio, and the like are as described above.

Since the present invention allows to improve renal disease, for example, to improve urinary protein and urinary protein/urinary creatinine ratio to a normal range, and to improve edema in a human patient having renal disease (for example, acute or chronic nephritis, and/or nephrotic syndrome), it is useful for enhancing or promoting the therapeutic effect on the disease (for example, suppressing the progression to renal failure) by using it in combination with a conventional treatment method for renal disease.

Examples of the conventional treatment methods for renal disease include diet (for example, energy restriction, salt restriction, protein restriction, and the like), potassium restriction, drug therapy (for example, corticosteroids, immunosuppressive drugs, anticoagulants, hypotensive drugs, antihyperlipidemic drugs, therapeutic drugs for hyperuricemia, erythropoietin preparations, and the like), and the above treatment methods can be implemented according to the stage of symptoms.

### EXAMPLES

The present invention will be described more specifically with reference to the following Examples, but the scope of the present invention is not limited by the Examples.

### Example 1

### Improvement of Urinary Protein and Urinary Protein/Urinary Creatinine Ratio in a Human Patient with Chronic Nephritis by Inhalation of Molecular Hydrogen

A woman in her 60s with chronic nephritis had 1+ to 3+ protein in the urine, sometimes 5+, and was told by doctors that she might need dialysis. This patient had a urinary protein of 3+, urinary protein determination of 286.5 mg/dl, urinary creatinine of 56.7 mg/dl, urinary protein/urinary creatinine ratio of 5.0 g/gCr, estimated glomerular filtration rate (eGFR) of 57.6, urea nitrogen (BUN) of 15.5 mg/dl, and serum creatinine of 0.79 mg/dl at the first visit, and was diagnosed as diabetic with a blood sugar level of 156 mg/dl, HbA1c of 8.0%, and glyco-albumin of 19.8%. She was taking a drug for lowering blood sugar, an insulin secretagogue, a hypotensive drug, and an anticoagulant.

Approximately one year after the first visit, she had a urinary protein of 1+, urinary protein determination of 57.9 mg/dl, urinary creatinine of 19.7 mg/dl, urinary protein/urinary creatinine ratio of 2.9 g/gCr, eGFR of 43.3, urea nitrogen (BUN) of 25.2 mg/dl, and serum creatinine of 1.02 mg/dl, and a blood sugar level of 122 mg/dl, HbA1c of 6.4%, and glyco-albumin of 14.2%, with moderate symptoms. From this point on, while taking the above medicaments prescribed by the doctor, the patient inhaled molecular hydrogen (hydrogen inhaler type MHG-2000α, hydrogen concentration 6 to 7.5%, MiZ Company Limited) about 2 to 3 hours per day, which she continued almost every day.

Approximately two and a half years after the first visit, her test results were a urinary protein of ±, urinary protein determination of 25.3 mg/dl, urinary creatinine of 24.1 mg/dl, urinary protein/urinary creatinine ratio of 1.05 g/gCr, eGFR of 52.0, urea nitrogen (BUN) of 22.1 mg/dl, serum creatinine of 0.91 mg/dl, and a blood sugar level of 181 mg/dl, HbA1c of 8.0%, and glyco-albumin of 24.7%, and the like. While renal function was mild to moderate, there was a tendency for improvement. However, the symptoms of diabetes seem to be getting worse.

Furthermore, the test results approximately three years after the first visit (about 2 years after the start of molecular hydrogen inhalation) were a urinary protein of 0.4+, urinary protein determination of 6.6 mg/dl, urinary creatinine of 27.3 mg/dl, urinary protein/urinary creatinine ratio of 0.24 g/gCr, eGFR of 45.9, urea nitrogen (BUN) of 21.5 mg/dl, and serum creatinine of 0.86 mg/dl, and a blood sugar level of 139 mg/dl, HbA1c of 6.5%, and glyco-albumin of 17.3%. An improvement in renal function and diabetic symptoms was observed, especially urinary protein (protein amount of about 0.1 g/day when the excreted urine volume is 1.5 L per day; normal levels: less than 0.2 g/day) and urinary protein/urinary creatinine ratio (0.24 g/gCr; normal levels: less than 0.3 g/gCr) were both in the normal range, and the doctor judged that dialysis was not required.

### Example 2

### Symptom Improvement in Patient with Renal Failure by Inhalation of Molecular Hydrogen

A 65-year-old woman developed renal failure from diabetes and has been on dialysis since 2006. Although she had used more than 20 kinds of drugs, her symptoms did not improve and she also had difficulty walking

Therefore, she started hydrogen gas inhalation in September 2019. Specifically, she did inhalations 3 to 4 hours per day using a hydrogen generator (model used: Jobs-a manufactured by MiZ Company Limited (hydrogen concentration: about 5%, other gas: air), amount of hydrogen gas generated: 200 ml/min).

She became able to walk 10 days after starting hydrogen inhalation. After dialysis, she usually had edema and feelings of heaviness, but 1 month after starting hydrogen inhalation, the edema decreased and her body felt lighter. Clearly, the improvement effect of hydrogen inhalation was observed.

### INDUSTRIAL APPLICABILITY

Since the present invention allows to improve renal disease, for example, to improve urinary protein and urinary protein/urinary creatinine ratio to a normal range, and to improve edema in a human patient having renal disease (for example, acute or chronic nephritis, renal failure, and/or nephrotic syndrome), it is useful for enhancing or promoting the therapeutic effect on the disease by using it in combination with a conventional treatment method for renal disease.

What is claimed is:

1. A method for preventing and/or improving a renal disease derived from human diabetes and/or hypertension in a human in need thereof, comprising administering to the human a composition comprising molecular hydrogen as an active ingredient.

2. The method according to claim 1, wherein the improvement of the renal disease is an improvement of acute or chronic nephritis, an improvement of renal failure, and/or an improvement of nephrotic syndrome.

3. The method according to claim 2, wherein the acute or chronic nephritis is selected from the group consisting of glomerulonephritis, interstitial nephritis, pyelonephritis, acute progressive glomerulonephritis, anti-glomerular basement membrane antibody nephritis, membranoproliferative glomerulonephritis, endocapillary proliferative glomerulonephritis, crescentic glomerulonephritis, and diabetic nephritis.

4. The method according to claim 2, wherein the improvement of the acute or chronic nephritis comprises improving urinary protein and urinary protein/urinary creatinine ratio to a normal range, or improving edema.

5. The method according to claim 4, wherein the normal range of urinary protein is less than 0.2 g/day, and the normal range of the urinary protein/urinary creatinine ratio is less than 0.3 g/gCr.

6. The method according to claim 1, wherein the composition is a gas composition comprising more than zero (0) and 18.5 vol % or less molecular hydrogen.

7. The method according to claim 1, wherein the composition is administered to the human by inhalation.

8. The method according to claim 1, wherein the composition is produced on the spot using a hydrogen generator.
